(19) United States
(12) Patent Application Publication
(10) Pub. No.: US 2007/0160494 A1
(43) Pub. Date: Jul. 12, 2007

(54) AUTOCLAVE SYSTEM USING RFID TAGS ON A CASE AND/OR INSTRUMENTS

(76) Inventor: Daniel L. Sands, Warsaw, IN (US)

Correspondence Address: TAYLOR & AUST, P.C., 142 SOUTH MAIN STREET, P. O. BOX 560, AVILLA, IN, 46710, US

(21) Appl. No.: 11/620,238

(22) Filed: Jan. 5, 2007

Related U.S. Application Data
(60) Provisional application No. 60/756,763, filed on Jan. 6, 2006.
(60) Provisional application No. 60/756,764, filed on Jan. 6, 2006.

(51) Int. Cl. A61L 2/24; A61L 2/07

(52) U.S. Cl. 422/26; 422/292; 340/539.1

(57) ABSTRACT

An autoclave system for sterilizing surgical instruments includes a sterilization case, at least one surgical instrument within the case, and at least one RFID tag associated with the case. Each RFID tag provides a wireless output signal representing a sterilization parameter associated with one or more surgical instruments. An autoclave receives the sterilization case, and includes a controller for controlling an autoclave cycle of the autoclave, dependent upon the wireless output signal from the RFID tag.

### CROSS REFERENCE TO RELATED APPLICATIONS

This is a non-provisional application based upon U.S. provisional patent application Ser. No. 60/756,763, entitled “RFID/WIRELESS AUTOCLAVE PARAMETER AND BACTERIAL SENSING DEVICES”, filed Jan. 6, 2006; and also based upon U.S. provisional patent application Ser. No. 60/756,764, entitled “AUTOCLAVE PREVENTION OF DISPOSABLES AND/OR HEAT SENSITIVE SURGICAL INSTRUMENTS”, filed Jan. 6, 2006.

### BACKGROUND OF THE INVENTION

1. Field of the Invention

The present invention relates to surgical instruments and storage cases, and, more particularly, to autoclaves for sterilizing surgical instruments.

2. Description of the Related Art

Typically, hospitals utilize large autoclaves and sterilize multiple containers or cases in a single autoclave cycle. The cycle time is determined by historical data and experienced guidelines from the hospital, manufacturers of medical equipment and other regulatory bodies. Problems with sterilization efficacy occur because hospitals batch multiple cases or containers within a sterilization cycle based on worst case scenario assumptions. These batch conditions continually change because the contents within a container change in each cycle. Autoclave cycles are run at worst case scenarios to ensure bacterial kill. This means autoclave cycle times run longer than optimal for sterility assurance.

Sometimes, containers or cases come out of the autoclave wet, i.e., they haven't stayed in the dry cycle long enough. This is called Wet Pack. Wet Packs cause hospitals considerable time and money because each item discovered to be wet can't be used and must be re-sterilized. Generally, large sets or heavy sets of instruments in a container or case or are contained in certain types of plastics or metal are more prone to wet packs. Orthopaedic implant and instrument systems generally fall into this category.

The problems of extended autoclave cycles and wet packs are not new. Chemical indicators and color indicators are used to show only if a containers has been through an autoclave cycle. Upon opening the container a chemical indicator will show if conditions were achieved for terminal sterilization. There is no known solution to solve the issue of wet packs.

Heat sensitive surgical instruments should only be sterilized using low temperature sterilization methods. Sometimes, items such as scopes, are mistakenly autoclaved, thus ruining a device that can cost up to $10,000. Conversely, disposable instruments are intended for single use. However, some disposable instruments are constructed of materials that will allow multiple autoclaves without damage and has prompted some hospitals to reuse these instruments to lower costs. Certain use specifications require durable material even though they are disposable. The reuse of these devices are against manufacturer recommendations affecting new sales opportunities.

Several heat sensitive sterilization products exist, but none that would prevent human error in placing the wrong device in the wrong sterilization process. Hospital procedures exist to prevent re-use of disposable items, but there is no fail safe device that will prevent re-use of an intended disposable item.

What is needed in the art is an autoclave system that will automatically set the autoclave cycle, and prevent autoclaving of disposable or heat sensitive instruments within a case.

### SUMMARY OF THE INVENTION

The invention in one form is directed to an autoclave system for sterilizing surgical instruments, including a sterilization case, at least one surgical instrument within the case, and at least one radio frequency identification (RFID) tag associated with the case. Each RFID tag provides a wireless output signal representing a sterilization parameter associated with one or more surgical instruments. An autoclave receives the sterilization case, and includes a controller for controlling an autoclave cycle of the autoclave, dependent upon the wireless output signal from the RFID tag.

The invention in another form is directed to a method of sterilizing surgical instruments, including the steps of: placing a surgical instrument within a sterilization case, the surgical instrument and/or the case coupled with an RFID tag; transmitting a wireless output signal from the RFID tag representing a sterilization parameter associated with the surgical instrument; setting an autoclave cycle for an autoclave, dependent upon the wireless output signal from the RFID tag; and autoclaving the case, including the surgical instrument, using the set autoclave cycle.

An advantage of the present invention is that the autoclave cycle is automatically set to provide adequate sterilization with minimum time and cost.

Another advantage is that the RFID tags provide a feedback system for setting the autoclave cycle.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawing, wherein:

FIG. 1 is a schematic illustration of an embodiment of an autoclave system of the present invention.

Corresponding reference characters indicate corresponding parts throughout the several views. The exemplification set out herein illustrates one embodiment of the invention, in one form, and such exemplification is not to be construed as limiting the scope of the invention in any manner.

### DETAILED DESCRIPTION OF THE INVENTION

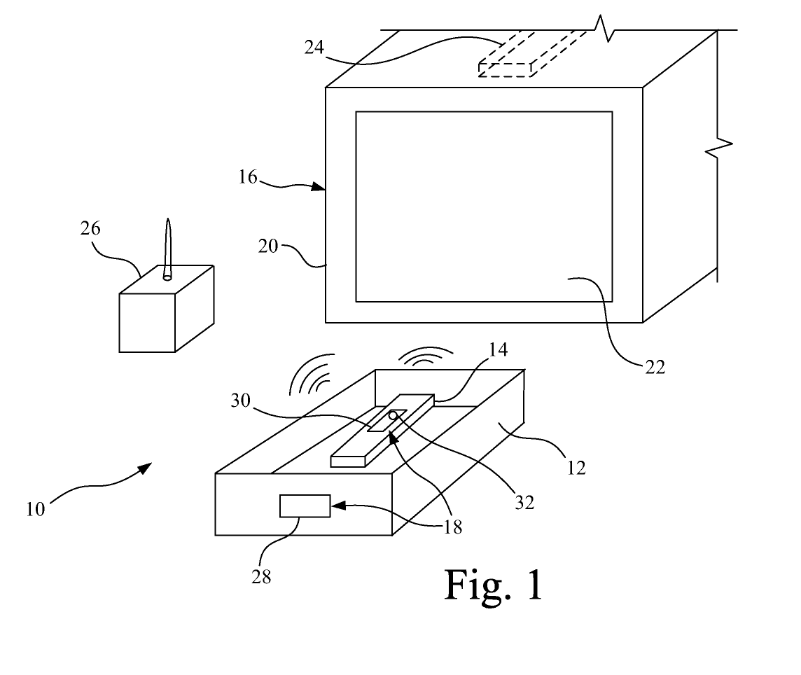

Referring now to the drawing, FIG. 1, there is shown a schematic illustration of an embodiment of an autoclave system 10 of the present invention. Autoclave system 10 generally includes a sterilization case 12, surgical instrument 14, autoclave 16, and one or more RFID tags 18.

Case 12 may be of any suitable size, shape and material to receive one or more instruments 14 therein. Case 12 typically includes a lid, hinged or unhinged, which covers case 12 after instrument(s) 14 are placed therein. The lid is not shown in the drawing for ease of illustration. The term “case”, as used herein, is intended to broadly mean any enclosure for surgical instruments that can be placed inside an autoclave, such as a case, container, housing, etc.

Surgical instrument 14 is shown schematically as a block in the figure, but can be any suitable surgical instrument which is to be sterilized within autoclave 16. For example, surgical instrument 16 can be, without limitation, a drill, reamer, implant, bone plate, scalpel, screws, etc. The term “instrument”, as used herein, is intended to broadly mean any workpiece used during surgery either to shape, cut or form tissue or bone, or implanted or otherwise remain within tissue or bone.

Autoclave 16 is of any suitable size and shape to receive one or more cases 12, and generally includes a housing 20, access door 22, and controller 24. Controller 24 may include electrical hardware and/or software suitable for controlling an autoclave cycle of autoclave 16 for adequate sterilization of case 12 and its contents, typically based upon temperature and time. Controller 24 includes or is coupled with a receiver or transceiver (not specifically shown) for receiving or receiving/transmitting wireless signals in the form of radio frequency signals, respectively. The embodiment of controller 24 shown in FIG. 1 is assumed to include an integral transceiver.

An optional base station 26 receives RF signals from an RFID tag 28 and/or 30 associated with case 12, and transmits a signal to the transceiver within controller 24. In the event base station 26 is not provided, then the transceiver within controller 24 receives the wireless output signals directly from RFID tag 28 and/or 30.

RFID tag 28 is coupled with the outside of case 12. RFID tag 28 is preferably a long range tag capable of transmitting a wireless RF signal over 50 feet, perhaps even approaching 300-400 feet. This is particularly helpful when using a base station 26 to communicate with autoclave 16, or for other wireless tracking of the whereabouts of case 12 within a building such as a hospital.

RFID tag 28 may be configured with onboard power, such as a long-life battery, allowing powering of RFID tag 28 upon a query by a wireless reader, whether local or remote. RFID tag 28 may also be configured to interrogate the one or more instruments within case 12, and transmit a corresponding output signal to the transceiver within controller 24 and/or to base station 26.

RFID tag 30 is coupled with the outside of instrument 14. RFID tag 30 is preferably a long range tag capable of transmitting a wireless RF signal over 50 feet, perhaps even approaching 300-400 feet, but may also be a short range tag capable of only transmitting a wireless RF signal a few feet. RFID tag 30 also optionally includes an onboard sensor 32 for sensing a physical parameter associated with the sterilization of instrument 14, such as a sensor to detect a presence of bacteria or water on instrument 14.

In the event that instrument 14 is a disposable or heat sensitive instrument, then RFID tag 30 provides a stop cycle output signal which prevents controller 24 from initiating the autoclaving process.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An autoclave system for sterilizing surgical instruments, comprising:
a sterilization case;
at least one surgical instrument within said case;
at least one RFID tag associated with said case, each said RFID tag providing a wireless output signal representing a sterilization parameter associated with at least one said surgical instrument; and
an autoclave for receiving said sterilization case, said autoclave including a controller for controlling an autoclave cycle of said autoclave, dependent upon said wireless output signal from said RFID tag.

2. The autoclave system of claim 1, wherein said output signal includes information pertaining to a required autoclave cycle for at least one said instrument.

3. The autoclave system of claim 1, wherein said at least one RFID tag includes an RFID tag mounted to said case.

4. The autoclave system of claim 3, wherein said RFID tag mounted to said case is a long range tag capable of transmission over 50 feet.

5. The autoclave system of claim 4, including a base station in communication with each of said RFID tag and said controller.

6. The autoclave system of claim 1, wherein said at least one RFID tag includes an RFID tag mounted to one of said instruments.

7. The autoclave system of claim 6, wherein said RFID tag mounted to said one instrument is a long range tag capable of transmission over 50 feet.

8. The autoclave system of claim 6, wherein said at least one instrument is one of a disposable instrument and a heat sensitive instrument, and said at least one RFID tag includes an RFID tag mounted to said instrument, said RFID tag providing a stop cycle output signal used by said controller to prevent an autoclave cycle.

9. The autoclave system of claim 6, wherein said RFID tag includes a sensor for sensing a physical parameter associated with said instrument.

10. The autoclave system of claim 9, wherein said physical parameter includes at least one of bacteria presence and water presence on said instrument.

11. The autoclave system of claim 1, wherein said wireless output signal is received and used directly by said controller to set said autoclave cycle.

12. The autoclave system of claim 1, wherein said autoclave cycle includes at least one of temperature and time.

13. A method of sterilizing surgical instruments, comprising:
placing a surgical instrument within a sterilization case, at least one of said surgical instrument and said case coupled with an RFID tag;
transmitting a wireless output signal from said RFID tag representing a sterilization parameter associated with said surgical instrument;
setting an autoclave cycle for an autoclave, dependent upon said wireless output signal from said RFID tag; and
autoclaving said case, including said surgical instrument, using said set autoclave cycle.

14. The method of sterilizing surgical instruments of claim 13, wherein said RFID tag is mounted to said case.

15. The method of sterilizing surgical instruments of claim 13, wherein said RFID tag is mounted to said surgical instrument.

16. The method of sterilizing surgical instruments of claim 15, wherein said surgical instrument is one of a disposable instrument and a heat sensitive instrument, and said RFID tag provides a stop cycle output signal to prevent an autoclave cycle.
